(19) United States
(12) Patent Application Publication
(10) Pub. No.: US 2019/0290836 A1
(43) Pub. Date: Sep. 26, 2019

(54) APPARATUS AND SYSTEM FOR GENERATING AT LEAST ONE INJECTION PARAMETER FOR A CONTRAST AGENT INJECTOR FOR COMPUTER TOMOGRAPHY IMAGING AND METHOD USING SAME

(71) Applicant: IMAGE Information Systems Europe GmbH, Rostock (DE)

(72) Inventor: Arpad Bischof, Rostock (DE)

(21) Appl. No.: 16/363,816

(22) Filed: Mar. 25, 2019

(30) Foreign Application Priority Data
Mar. 26, 2018 (DE) 102018107080.2

(51) Int. Cl. A61M 5/00; A61M 31/00; A61B 6/00; A61B 8/06

(52) U.S. Cl. CPC A61M 5/007; A61M 31/005; A61M 5/142; A61B 8/06; A61B 6/504; A61B 6/481

(57) ABSTRACT

The disclosure concerns an apparatus for generating at least one injection parameter for a contrast agent injector. The apparatus includes a first data interface for receiving patient data and a second data interface for connection to a contrast agent injector. The apparatus also includes a data processing unit for generating at least one injection parameter in dependence on patient data received by way of the first data interface and for outputting the injection parameter at the second data interface. The disclosure further concerns a system comprising such an apparatus as well as a method therewith.

### BACKGROUND

### Technical Field

The disclosure concerns the field of computer tomography. In computer tomography a computer tomography scanner is used to record pictures which are also referred to as images of regions of interest of a human body like regions in which there are certain organs. For that purpose cross-sectional images are produced by means of X-rays passing through the body, and those images are assembled together with a computer to form a scan recording.

### Description of the Related Art

Usually for investigation purposes a contrast agent, in particular an iodine-bearing contrast agent, is intravenously injected into a patient. After the injection the contrast agent passes through the blood vessels. The property of the contrast agent that it absorbs X-rays to a greater or lesser degree than the blood which is otherwise flowing in the blood vessels makes it possible to represent regions in which the contrast agent is present as being lighter or darker in relation to the other regions.

It is known that complications can arise by virtue of differing reactions on the part of a body into which the contrast agent is injected. Therefore, prior to a computer tomography investigation different medical investigations are carried out in order already thereby to minimize a large number of possible complications.

In dependence on a region or a plurality of regions to be imaged in a body the doctor performing the treatment additionally predetermines injection parameters for a contrast agent injector in order on the one hand to take an adequate amount of the contrast agent into the region of the body that is of interest at the desired moment in time and on the other hand to minimize as far as possible the amount of contrast agent introduced in order to avoid complications. Injection parameters include in particular an amount of contrast agent, the flow rate at which the contrast agent is administered, and a moment in time or a period in relation to producing the image with the computer tomography scanner. Producing the image with the computer tomography scanner is also referred to as image acquisition. In particular the moment in time is a particularly important factor as the contrast agent firstly has to flow from the injection location to the region of interest for the desired imaging before imaging is carried out with the computer tomography scanner.

Accordingly there is only a certain time window after injection of the contrast agent for computer tomography imaging with the computer tomography scanner. If therefore imaging with the computer tomography scanner is carried out before or after that time window there is then the risk of a low-contrast image which is limited or cannot be correctly used. In such a situation a further imaging operation would have to be carried out, for which contrast agent would have to be injected afresh. In order very substantially to avoid or even exclude repeated imaging the common practice is to select the time window to be longer than is absolutely necessary, the result of this being that an amount of contrast agent, that is more than actually required for the imaging operation, is injected.

As already stated hereinbefore however the aim is to keep the amount of contrast agent as low as possible, in which respect a precise quantity in respect of the injection parameters is therefore determined by the treatment doctor based on patient data and the past experience of the treatment doctor. In particular younger staff have no or only slight past experience so that they can determine the injection parameters only in very broad terms based on textbook knowledge. By virtue of a large number of individual prerequisites in respect of the body parts to be diagnosed however the injection parameters can only be roughly ascertained even by experienced staff so that—to avoid repeated scanning imaging—a substantially excessive amount of contrast agent is generally injected. That correspondingly also results in a comparatively high number of complications in patients who have received an injection of contrast agent, in which respect that high number could be reduced if the optimum amount of contrast agent is used in conjunction with an optimum moment in time for the injection. People of lower body weight typically receive an overdose of contrast agent while overweight people frequently receive an underdose.

### BRIEF SUMMARY

One or more embodiments may combat the problems in the state of the art and in particular to determine injection parameters which are improved over known methods for the injection of a contrast agent in a computer tomography imaging operation.

One embodiment is directed to an apparatus for generating at least one injection parameter for a contrast agent injector. The contrast agent injector is also referred to as the contrast agent pump.

The apparatus includes a first data interface which is implemented by one or more physical data interfaces, and a second data interface which is implemented by one or more physical data interfaces. The first data interface serves to receive patient data and the second data interface serves for connection to a contrast agent injector. The term contrast agent injector is used here to denote a contrast agent injection unit itself as well as a contrast agent injector control means for the contrast agent injection unit, for example by means of a computer.

Provided is a data processing unit for generating injection parameters and for outputting the injection parameters at the second data interface. The data processing unit is adapted to determine the injection parameters in dependence on patient data which can be received by way of the first data interface.

The apparatus thus represents an interface between a contrast agent injector and a source of patient data in order to determine suitable injection parameters from the patient data and to transmit them to the contrast agent injector. Unlike a doctor performing the treatment the apparatus can automatically take account of a large amount of patient data in generating the injection parameters so that optimized injection parameters, for example in relation to an amount of contrast agent or a time for the contrast agent injection, can be substantially more precisely determined. A reduction in the amount of contrast agent can be achieved by such optimization. A further advantage is that errors in determining injection parameters due to human misjudgments can be excluded.

Preferably the apparatus is also adapted to receive information about the region of interest in a patient, in respect of which computer tomography imaging is to be effected, for example in addition by way of the first data interface or by way of input means of the apparatus.

According to a first embodiment the first data interface is adapted to be connected to a measuring device for detecting physiological parameters, in particular a pulse and/or blood pressure measuring device and/or a thermometer, preferably a body temperature probe, and/or an oximeter, preferably a pulse oximeter, wherein the patient data include physiological parameters like pulse and/or blood pressure measurement values and/or temperature values and/or oxygen saturation values. Those physiological parameters are preferably transmitted in real time or with a short time delay, for example with a maximum time delay of 30 seconds, to the apparatus. Preferably therefore current patient data, more specifically physiological parameters which are also referred to as circulation parameters, are available to the apparatus so that they can be taken into account when generating the injection parameters. By virtue thereof, besides other patient data, it is also possible to take account of a currently prevailing circulation state of the patient in determining and generating the injection parameters.

It was accordingly recognized that a person can have a comparatively higher pulse rate even without physical effort, even if that person at the start had a substantially constant resting pulse, precisely when the person is exposed to particular situations which also include preparation for a computer tomography scan. That increased pulse rate accordingly also results in faster progression of the contrast agent through the blood vessels. If now, immediately prior to carrying out a scan with a computer tomography scanner, there is the possibility in regard to determining the injection parameters to directly take account of a physiological parameter like the currently prevailing pulse rate and/or blood pressure then the amount of contrast agent and the time of injection can be determined still more precisely. In that way the amount of contrast agent can be further reduced, more specifically particularly by precisely determining the time of the injection of contrast agent. It was also realized that the circulation changes in the case of seriously ill patients due to reduced oxygen saturation and fever. That change can also be taken into consideration.

In accordance with a further embodiment the first data interface is also adapted to be connected to a patient database in order to retrieve further patient data in order to take those into account when generating the injection parameters. In that respect patient data from the patient database include for example previous illnesses, diagnoses, laboratory parameters or previously stored physiological parameters like oxygen saturation or the body temperature during a previous scan, sex, height, weight, age and/or body mass index of various patients.

The apparatus is therefore adapted to assess a plurality of or all available stored patient data in a patient database in accordance with predetermined rules in the apparatus, and to take them into account in generation of the injection parameters. In that way highly individual generation of injection parameters for a patient is possible.

In a further embodiment the first data interface includes a wireless interface, in particular with a proprietary protocol. A measuring device for detecting physiological parameters can thus be for example wirelessly connected to the apparatus in order to receive physiological parameters like pulse rate and/or blood pressure measurement values, body temperature or oxygen saturation, from the patient who has already been brought into position in preparation for the scan. A pulse rate and/or blood pressure measuring device can accordingly be worn by the patient throughout the entire investigation without limitations due to a cable so that even during and after the investigation physiological parameters like pulse rate and/or blood pressure measurement values can be recorded and/or stored for further assessments.

According to a further embodiment the injection parameters include at least one value for an amount of contrast agent and an injection time and/or injection period and/or preferably a flow rate and/or a flow profile. By way of example a flow profile which can also be referred to as a flow-time profile includes a flow of 5 ml/s for 5 seconds, 4 ml/s for a further 3 seconds and 3 ml/s for a further 10 seconds.

By virtue of the output of the injection parameters by way of the second data interface the staff can perform a further examination of the data and possibly also individually adapt the parameters before they are then actually applied in the contrast agent injector. A concentration plateau of a uniform level can be implemented by a value for an amount of contrast agent and an injection time and/or injection period and/or preferably a flow rate and/or a flow profile.

In accordance with a further embodiment the apparatus has a third data interface to receive data from a computer tomography scanner. The third data interface is implemented by one or more physical data interfaces. The apparatus is preferably adapted to assess those data, in particular the image data, but also to receive status information in respect of the investigation, like for example investigation started, investigation suspended, investigation terminated and so forth.

In addition the data can be updated directly into a patient database, a patient file and/or a documentation system, for example for contrast agent. That permits automatic quantification of the desired image contrast after the investigation in order thereby to assess the injection parameters generated. Alternatively or additionally the third data interface serves to send control signals to the computer tomography scanner, in particular for carrying out a scan.

The control signals can include for example a further moment in time in relation to the time for contrast agent injection so that the apparatus is overall adapted to control the contrast agent injector, even without staff, on the basis of the patient data which were received by way of the first data interface with the second data interface, and the computer tomography scanner with the third data interface. Fully automatic implementation of a scan, that is to say without manual intervention, is thus possible with the apparatus.

In a further embodiment the second data interface or a fourth data interface which is implemented by one or more physical data interfaces is adapted to receive from the contrast agent injector data like injection parameters carried out in an investigation. In the situation where the staff therefore also adapts the previously generated and output injection parameters of the apparatus for a treatment which is performed later the implemented injection parameters in accordance with this embodiment are made available to the apparatus again in order to take them into account for example for later parameter generating operations. In addition the data received from the contrast agent injector can be associated with the patient being investigated. In that way the data can for example be automatically updated into a patient file.

In particular that is advantageous in order to store the implemented injection parameters together with the assessed image contrast of the picture produced in that case in order to recognize whether for example adaptation has resulted in a comparatively good or poor result, in order possibly to take account of adaptation in the apparatus in subsequent investigations.

In a further embodiment the data processing unit is adapted to adapt the generation of the injection parameters themselves, in dependence on the data acquired by way of one or more data interfaces, that is to say for example for generating rules applied, instructions and so forth, in particular by machine learning means.

By way of example therefore assessed images which are obtained from the computer tomography scanner are evaluated together with the injection parameters actually involved, and on the basis of the assessment the generation of injection parameters can be adapted for later parameter generating operations. Automatic improvement in the scanned images is thus possible and the generation of injection parameters is thus still further optimized.

Provided is a system having an apparatus according to one of the above-described embodiments. In addition the system includes a measuring device which is or can be connected, in particular wirelessly, to the apparatus by way of the first data interface of the apparatus, for detecting physiological parameters, for example a pulse rate and/or blood pressure measuring device and/or a thermometer like for example a body thermometer, or a body temperature probe, and/or an oximeter, like for example a pulse oximeter.

According to an embodiment of the system it includes a contrast agent injector connected by way of the second interface of the apparatus.

According to a further embodiment of the system the measuring device is a wrist or ankle measuring device. The measuring device can accordingly be arranged to measure at the wrist and/or ankle of a patient.

In that way it is possible for example to provide for the transmission of the physiological parameters to the apparatus during an investigation without the imaging scans of any body part with the computer tomography scanner being influenced by the measuring device.

Provided is a method of generating an image with a computer tomography scanner. The method is preferably carried out with an apparatus according to one of the above-described embodiments or a system according to one of the above-described embodiments. In that situation firstly patient data are obtained by way of a first data interface and at least one injection parameter for a contrast agent injector is generated with a data processing unit in dependence on the patient data. The at least one injection parameter is then output at a second interface.

Optimum determination of injection parameters for a contrast agent injector is thus possible in an automated procedure.

According to an embodiment of the method it also includes actuation of a contrast agent injector with the generated and output injection parameters or with modified generated and output injection parameters and/or actuation and/or reception of data from a computer tomography scanner. Thus the method permits completely autonomous implementation of a scan with a computer tomography scanner, including execution of a contrast agent injection. In addition however intervention by operating staff like for example a doctor carrying out the treatment is made possible by adaptation of the injection parameters.

According to a further embodiment of the method the data from a computer tomography scanner include a computer tomography image, wherein the computer tomography image is assessed with the apparatus by means of image processing software and at least the assessment with the injection parameters used is stored. Storage is preferably effected in a database, to which access can be had by way of a network or an Internet connection.

In dependence on the injection parameters therefore the computer tomography image is assessed and stored so that the quality of the previously generated injection parameters can be indirectly assessed.

According to a further embodiment the method includes the generation of at least one injection parameter with the data processing unit in dependence on the patient data and additionally in dependence on at least one previously stored assessment. Experimental values which were obtained by assessment of the computer tomography imaging scans which were performed thus serve to provide an improvement in the generation of the injection parameters.

According to a further embodiment of the method the detection of patient data includes measurement of physiological parameters like pulse rate and/or blood pressure data and/or body temperature values and/or body oxygen saturation values with a measuring device for detecting physiological parameters like a pulse rate and/or blood pressure measuring device and/or a thermometer, preferably a body temperature probe, and/or an oximeter, in particular a pulse oximeter, and transmission of the physiological parameters from the measuring device to an apparatus for generating the injection parameter. Currently applicable patient data like circulation parameters can thus be taken into account when generating the injection parameter.

According to a further embodiment of the method detection of patient data includes access to a patient database in order to retrieve further patient data of a patient to be treated from the patient database, more specifically from an electronic patient file for the respective patient.

A plurality of further patient data can therefore be taken into consideration in generating the injection parameter so that an optimum individual injection parameter can be generated.

### BRIEF DESCRIPTION OF THE SEVERAL VIEWS OF THE DRAWINGS

Further configurations will be apparent from the embodiments by way of example which are described in greater detail with reference to the Figures.

In the Figures:

FIG. 1 shows an embodiment of the apparatus according to the disclosure,

FIG. 2 shows an embodiment of a system with the apparatus, and

FIG. 3 shows steps in the method according to an embodiment.

### DETAILED DESCRIPTION

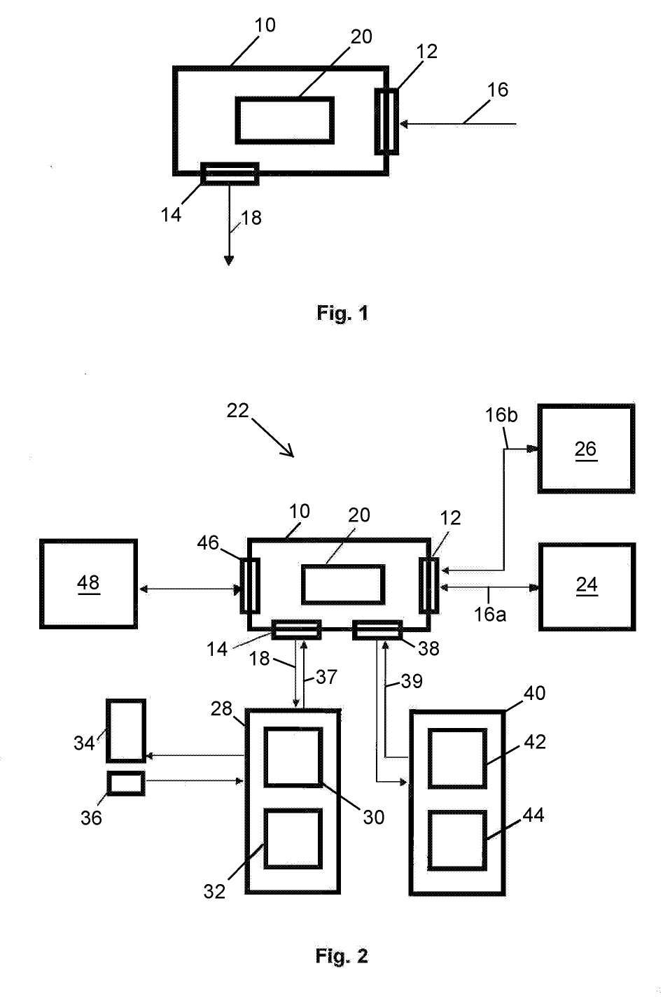

FIG. 1 shows an apparatus 10 for generating at least one injection parameter for a contrast agent injector according to a first embodiment. The apparatus 10 includes a first data interface 12 and a second data interface 14. The first data interface 12 is adapted to receive patient data 16 and the second data interface 14 is adapted to be connected to a contrast agent injector. The second data interface 14 is also adapted to output at least one injection parameter 18 to a connected contrast agent injector. An injection parameter 18 includes for example an injection time and/or an injection period as well as an amount of contrast agent and/or a flow rate of a contrast agent and/or a flow profile.

The apparatus 10 further includes a data processing unit 20 adapted to generate at least one injection parameter 18 in dependence on the patient data 16 received by way of the first data interface 12.

FIG. 2 shows the apparatus 10 for generating at least one injection parameter 18 within a system 22 according to an embodiment of the disclosure. Besides the apparatus 10 the system includes a measuring device for detecting physiological parameters 24, which here is a pulse rate and/or blood pressure measuring device and outputs patient data 16, more specifically physiological parameters 16a, more specifically here pulse rate and/or blood pressure measurement values, and feeds same to the first data interface 12 of the apparatus.

In addition there is a patient database 26 also connected to the apparatus 10 by way of the first data interface 12 so that further patient data 16b like for example previous illnesses, diagnoses, sex, height, weight, age and/or body mass index can be output. The patient data 16, more specifically including currently measured patient data like the physiological parameters 16a, as well as the further patient data 16b, are then passed to a data processing unit 20 and with the data processing unit 20 at least one injection parameter 18 for a contrast agent injector 28 is generated and output by way of the second data interface 14.

In this arrangement the contrast agent injector 28 includes a contrast agent injector actuating means 30 as well as a contrast agent injection unit 32. The contrast agent injector actuating means 30 is preferably adapted to optically represent the injection parameters 18 with a display 34 and also has input means 36 with which the injection parameters 18 can be modified or adapted. In addition the contrast agent injector 28 is adapted on the basis of the generated injection parameters 18 or the adapted generated injection parameters to actuate the contrast agent injection unit 32. The injection parameters 37 used in that actuating means 30, in accordance with this preferred embodiment, are made available to the apparatus 10 by way of the second data interface 14.

In addition the apparatus 10 includes a third data interface 38 to which a computer tomography scanner 40 is connected. The computer tomography scanner 40 also includes a computer tomography scanner actuating means 42 as well as a computer tomography scanning unit 44. The third data interface 38 is adapted to control the computer tomography scanner 40 or at least supply moments in time for the implementation of a scan. In addition the third data interface 38 is adapted to receive data 39, in particular including an image from the computer tomography scanner 40, with picture data.

The apparatus 10 is further connected by way of a further data interface 46 which is preferably connected to a network or an Internet connection to a database 48 in which the received images as well as the injection parameters 37 implemented are stored together with an assessment effected by the data processing unit 20. Data from that database 48 can also be received by way of the further data interface 46 in order to optimize the generation of injection parameters 18 with the data processing unit 20 by machine learning.

In addition the apparatus 10 includes input means (not shown) in order to predetermine a region of interest, for which a computer tomography scan is wanted.

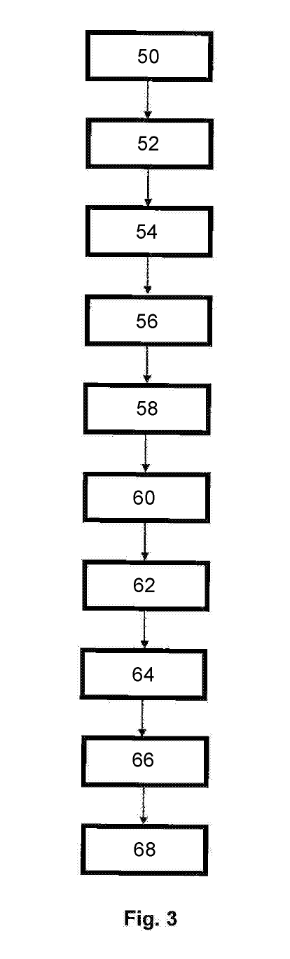

FIG. 3 shows an embodiment of the method according to the disclosure.

In a first step 50 patient data 16 of a selected patient are loaded or retrieved from a patient database 26 into the apparatus 10 by way of a first data interface 12.

In a step 52 a region for which the computer tomography scan is to be carried out is predetermined for the apparatus 10. Such a region is for example a part of the body or an organ of a patient. Presetting of the region of interest is effected for example by retrieval by a computer tomography scanner control means 42 or by input means connected to the apparatus.

In a step 54 current physiological parameters 16a like for example pulse rate and/or blood pressure measurement values are then received from a measuring device 24 and injection parameters 18 are determined in dependence on all received data in the step 56. Those injection parameters 18 are fed to a contrast agent injector 28 in a step 58. The contrast agent injector 28 represents the injection parameters 18 on a display 34 in a step 60 and in a step 62 waits until manual input is effected in order to adapt the injection parameters 18 or confirm same. Thereupon in a step 64 the apparatus 10 waits until an imaging scan has been carried out with the computer tomography scanner 40 and receives the image from the computer tomography scanner 40. That image is assessed in a step 66 and stored together with the injection parameters 18 used received from the contrast agent injector 28 in the database 48 in a step 68.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus for generating at least one injection parameter for a contrast agent injector, the apparatus comprising:
a first data interface configured to receive patient data;
a second data interface configured to connect to a contrast agent injector; and
a data processing unit configured to generate at least one injection parameter in dependence on patient data received by way of the first data interface and to output the injection parameter at the second data interface.

2. The apparatus according to claim 1 wherein the first data interface is adapted to be connected to one or more measuring devices.

3. The apparatus according to claim 1 wherein the first data interface is configured to be connected to a patient database to retrieve or receive further patient data, wherein the further patient data includes previous illnesses, diagnoses, sex, height, weight, age and/or body mass index values.

4. The apparatus according to claim 1 wherein at least one of the first data interface and the second data interface includes a wireless interface with a proprietary protocol.

5. The apparatus according to claim 1 wherein the at least one injection parameter includes a value for at least one of: an amount of contrast agent, an injection time, injection period, a flow rate, and a flow profile of a contrast agent.

6. The apparatus according to claim 1 further comprising a third data interface configured to receive data from a computer tomography scanner and to assess the received data, wherein the received data includes image data, wherein the third data interface is configured to send control signals to the computer tomography scanner, to cause an imaging scan.

7. The apparatus according to claim 1 wherein the second data interface or a fourth data interface is configured to receive data that includes implemented injection parameters, from the contrast agent injector.

8. The apparatus according to claim 1 wherein the data processing unit is configured, in dependence on data received by a data interface chosen among the first data interface, the second data interface, and a third data interface to adapt the generation of the at least one injection parameter by machine learning means.

9. A system comprising:
the apparatus according to claim 1; and
a measuring device configured to be connected to the apparatus by way of the first data interface, and configured to detect physiological parameters, wherein the physiological parameters include a pulse rate and/or blood pressure.

10. The system according to claim 9 further comprising a contrast agent injector connected by way of the second data interface.

11. The system according to claim 9 wherein the measuring device is at least one device chosen among: a wrist pulse rate measuring device, an ankle pulse rate measuring device, a blood pressure measuring device for pulse rate, a blood pressure measurement at for a wrist and/or ankle, a thermometer, a body thermometer, a body temperature probe, an oximeter, and a pulse oximeter.

12. A method of generating an image recorded with a computer tomography scanner, the method comprising:
detecting patient data by way of a first data interface;
generating an injection parameter for a contrast agent injector with a data processing unit in dependence on the patient data; and
outputting the injection parameter at a second data interface.

13. The method according to claim 12 further comprising at least one step chosen among:
actuating a contrast agent injector with the output injection parameter or a modified generated injection parameter;
actuating data from a computer tomography scanner; and
receiving data from the computer tomography scanner.

14. The method according to claim 13 wherein the data received from the computer tomography scanner includes a computer tomography scanning image, the method further comprising assessing the computer tomography scanning image using image processing software, and storing at least the assessment with the implemented injection parameter.

15. The method according to claim 12 wherein the generating the injection parameter comprises generating the injection parameter in dependence on the patient data and in dependence on at least one previously stored assessment.

16. The method according to claim 12 wherein the generated injection parameter is represented by the contrast agent injector on a display and the generated injection parameter can be modified by way of a user interface.

17. The method according to claim 12 wherein detecting the patient data includes retrieving physiological parameters from a measuring device and transmitting the physiological parameters from the measuring device to the apparatus.

18. The method according to claim 12 wherein detecting the patient data includes accessing a patient database and retrieving further patient data.

19. The apparatus according to claim 2, wherein the one or more measuring devices are configured to detect physiological parameters and the patient data, wherein the patient data includes pulse rate and blood pressure measurement values.

20. The method according to claim 14 wherein storing at least the assessment with the implemented injection parameter comprises storing at least the assessment with the implemented injection parameter in a patient database or a documentation system through a network or an internet connection.
